(19) United States
(12) Patent Application Publication
(10) Pub. No.: US 2016/0008224 A1
(43) Pub. Date: Jan. 14, 2016

(54) CONNECTOR FOR A CONTAINER INCLUDING A MEDICINAL ACTIVE INGREDIENT

(71) Applicant: Fresenius Kabi Deutschland GmbH, Bad Homburg (DE)

(72) Inventors: Ismael Rahimy, Friedberg (DE); Torsten Brandenburger, Reichelsheim (DE)

(21) Appl. No.: 14/797,379

(22) Filed: Jul. 13, 2015

Related U.S. Application Data
(63) Continuation of application No. 13/577,007, filed on Sep. 12, 2012.
(60) Provisional application No. 61/302,192, filed on Feb. 8, 2010.

(30) Foreign Application Priority Data
Feb. 8, 2010 (EP) 10152921.2

(51) Int. Cl. A61J 1/20; A61J 1/14; A61M 39/22

(52) U.S. Cl. CPC A61J 1/201; A61M 39/221; A61J 1/1475; A61J 1/1481

(57) ABSTRACT

The invention relates to a connector for containers containing medicinal active ingredient, which connector makes it possible to transfer active ingredient from one container into another container.

The connector (1, 1′) according to the invention comprises a first connection region (3, 3′) for the connection of a first container (2, 2′), a guide element (4, 4′), a piercing element (5, 5′) and a partition wall (6), wherein the guide element (4, 4′) has a duct (7) designed for transferring a medicinal active ingredient, the piercing element (5, 5′) is arranged, at least in certain sections, in the duct (7) and is axially movably guided within the duct (7) by the guide element (4, 4′), and, by connecting a container (2, 2′) in the first connection region (3, 3′), the piercing element (5, 5′) can be moved from a starting position, in which the piercing element (5, 5′) does not open the partition wall (6), into an end position, in which the piercing element (5, 5′) opens the partition wall (6) in order to transfer a medicinal active ingredient.

### TECHNICAL FIELD

The invention relates to a connector for containers containing medicinal active ingredient, which connector makes it possible to transfer active ingredient from one container into another container.

### PRIOR ART

EP 2 095 805 A2 discloses a connector for containers containing medicinal active ingredient, which connector comprises a first connection region for the connection of a glass vial, a second connection region for the connection of a flexible bag and an axially movable piercing element taking the form of a cannula which is sharpened on two sides. The piercing element is mounted in an axially displaceable manner in a first housing section of a housing of the connector. The first housing section is mounted in a telescopically displaceable manner on a second housing section. In the starting position of the connector, the first housing section and the second housing section are pulled apart. Sy connecting a glass bottle in the first connection region and by exerting a pressure on the glass bottle, the first housing section is displaced with respect to the second housing section. A membrane arranged in the neck of the glass bottle and closing the glass bottle strikes one end of the piercing element. Pushing forward the glass bottle takes along the piercing element until it strikes by its other end against a membrane enclosing the flexible bag. Further pushing forward of the glass bottle causes both the membrane belonging to the flexible bag and also the membrane of the glass bottle to be pierced by the piercing element. A limit stop for the movement of the piercing element limits the movement of the piercing element and ensures that not only the membrane belonging to the bag but also the membrane of the glass bottle is opened.

A disadvantage of this connector is that it comprises parts which are movable with the two housing sections and which have to be sealed with respect to one another. Furthermore, the connector comprises a large number of elements which make the production of the connector complicated and expensive.

### BRIEF DESCRIPTION OF THE INVENTION

The connector according to the invention for containers containing medicinal active ingredient comprises a first connection region for the connection of a first container, a guide element, a piercing element and a partition wall, wherein the guide element has a duct designed for transferring a medicinal active ingredient, the piercing element is arranged, at least in certain sections, in the duct and is axially movably guided within the duct by the guide element, and, by connecting a container, the piercing element can be moved from a starting position, in which the piercing element does not open the partition wall, into an end position, in which the piercing element opens the partition wall in order to transfer a medicinal active ingredient.

The fact that the guide element forms both the duct through which an active ingredient can be transferred and also forms a mount in which the piercing element can be displaced in an axially movable manner means that the guide element can adopt a dual function. Consequently, the number of elements of the connector can be reduced and the connector can be produced cost-effectively. It is also possible in this manner to reduce the number of interfaces and to improve the handling of the connector. The connector according to the invention makes it possible in particular to transfer the medicinal active ingredient without risk of contamination.

The containers which can be connected or attached by the connector may be both closed containers, for example glass bottles, plastic bottles or bags, in particular flexible bags, and “open” containers, for example catheters or other lines, as are used, for example in infusion, transfusion, clinical nourishment, oncology, dialysis or other medical fields. The medicinal active ingredient which can be transferred from one container to the other container by means of the connector may be a liquid or else a powder, for example.

The partition wall may be a flexible partition wall, for example consisting of an elastic material, in particular in the form of a membrane, or else, which is preferred, a rigid partition wall which is broken open or pierced by the piercing element.

In a preferred embodiment, the piercing element and the guide element are designed in such a way that a clearance for transferring a medicinal active ingredient remains between an outer face of the piercing element and an inner face, forming the duct, of the guide element. In an alternative embodiment, the piercing element and the guide element are designed in such a way that the medicinal active ingredient is additionally or exclusively transferred by means of the piercing element. For the respective purpose, the piercing element can be designed to be solid or as a hollow body. A combination of hollow body and solid body is also possible in principle. The profile of the piercing element preferably changes along its axis, with the end section facing the partition wall being designed to break open or pierce the partition wall, and the end section facing the container to be connected being designed in such a way that a pressure can be exerted on the end section by means of the container, which pressure can be used to press the piercing element against the partition wall. For this purpose, the end section facing the partition wall may, for example, be designed to taper with a linear or cross-shaped profile, while the other end section may be designed with, for example, a cross-shaped or round profile so as to form a bearing surface. In the case where the piercing element is designed as a hollow body, the end section facing the partition wall may be designed with a faceted finish or have a swaged tip.

In a preferred embodiment, the guide element and the partition wall are formed in one part, being integral in a particularly preferred embodiment.

In a further preferred embodiment, the connector comprises a housing forming the first connection region. Housing, guide element and partition wall are preferably formed in one part, particularly preferably integrally. The one-part and possibly integral formation of a plurality of functional elements makes it possible to reduce the number of parts of the connector, and the production costs can be reduced.

In a further preferred embodiment, piercing element, guide element, partition wall and/or housing are produced from a plastic, preferably polypropylene (PP) or a blend of polypropylene and styrene/ethylene-butylene/styrene (SEBS). In particular, piercing element, guide element, partition wall and/or housing may be injection moldings. Alternatively, the piercing element can be produced from, for example, polycarbonate (PC) or polystyrene (PS).

In a further preferred embodiment, the guide element comprises an end section which is designed to pierce a membrane of a container which can be connected into the first connection region, for example to pierce a rubber stopper of a glass vial. The guide element thus comprises a further function. A further element which is intended for piercing a membrane can be dispensed with in this manner.

In a further preferred embodiment, the first connection region is designed as a Luer connection of a Luer lock, in particular as a male part. In this case, the guide element is formed partially or completely by the central cone of the Luer connection. By connecting the female part, and on screwing the connection, the piercing element is preferably pressed from its starting position into the end position, in which the partition wall is opened. The connection of the counterpart of the Luer connection can thus be connected with an opening of the partition wall, and an additional movement sequence can thus be dispensed with. Moreover, the risk of contamination is reduced.

Further advantageous embodiments of the invention form the subject matter of the dependent claims.

The invention will be explained in greater detail with reference to exemplary embodiments, which are depicted by means of a plurality of figures.

### BRIEF DESCRIPTION OF THE FIGURES IN THE DRAWING

In the drawing:

FIG. 1 shows a longitudinal section through a first embodiment of a connector according to the invention,

FIG. 2 shows a section through the connector shown in FIG. 1 along the section line C-C,

FIG. 3 shows a first perspective view of the connector shown in FIG. 1,

FIG. 4 shows a second perspective view of the connector shown in FIG. 1,

FIG. 5 shows a portion of the piercing element of the connector shown in FIG. 1,

FIG. 6 shows a perspective view of the connector shown in FIG. 1, cut away along the longitudinal axis, in a starting state,

FIG. 7 shows a perspective view of the connector shown in FIG. 1, cut away along the longitudinal axis, before connecting a glass vial,

FIG. 8 shows a perspective view of the connector shown in FIG. 1, cut away along the longitudinal, axis, with the glass vial in a docking position,

FIG. 9 shows a perspective view of the connector shown in FIG. 1, cut away along the longitudinal axis, with the glass vial in the connected end position,

FIG. 10 shows a perspective view of the first connection region of the connector shown in FIG. 1,

FIG. 11 shows a side view of a second embodiment of a connector according to the invention with a Luer female part connected to the connector,

FIG. 12 shows a perspective view of the connector shown in FIG. 11 with a Luer female part,

FIG. 13 shows a plan view of the connector shown in FIG. 11,

FIG. 14 shows a longitudinal section through the connector shown in FIG. 11 with a Luer female part,

FIG. 15 shows an enlarged representation of the detail A depicted in FIG. 14,

FIG. 16 shows a perspective view of the connector shown in FIG. 11, cut away along the longitudinal axis, before connecting the Luer female part,

FIG. 17 shows a perspective view of the connector shown in FIG. 11, cut away along the longitudinal axis, with a connected Luer female part, and

FIG. 18 shows a perspective view of the piercing element of the connector shown in FIG. 11.

### DESCRIPTION OF THE TYPES OF EMBODIMENT

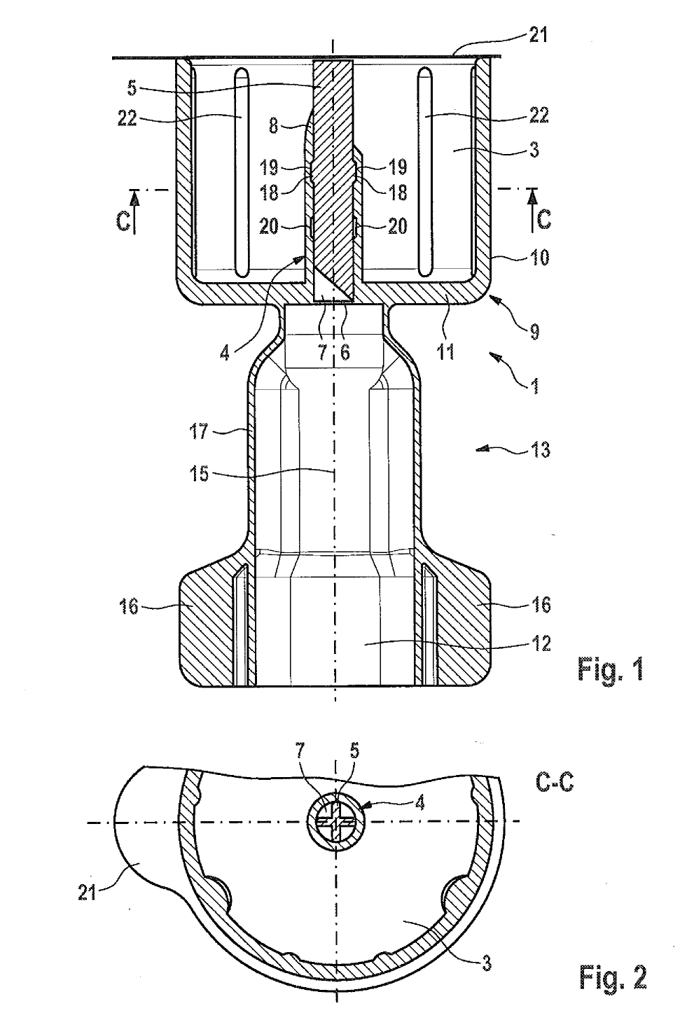

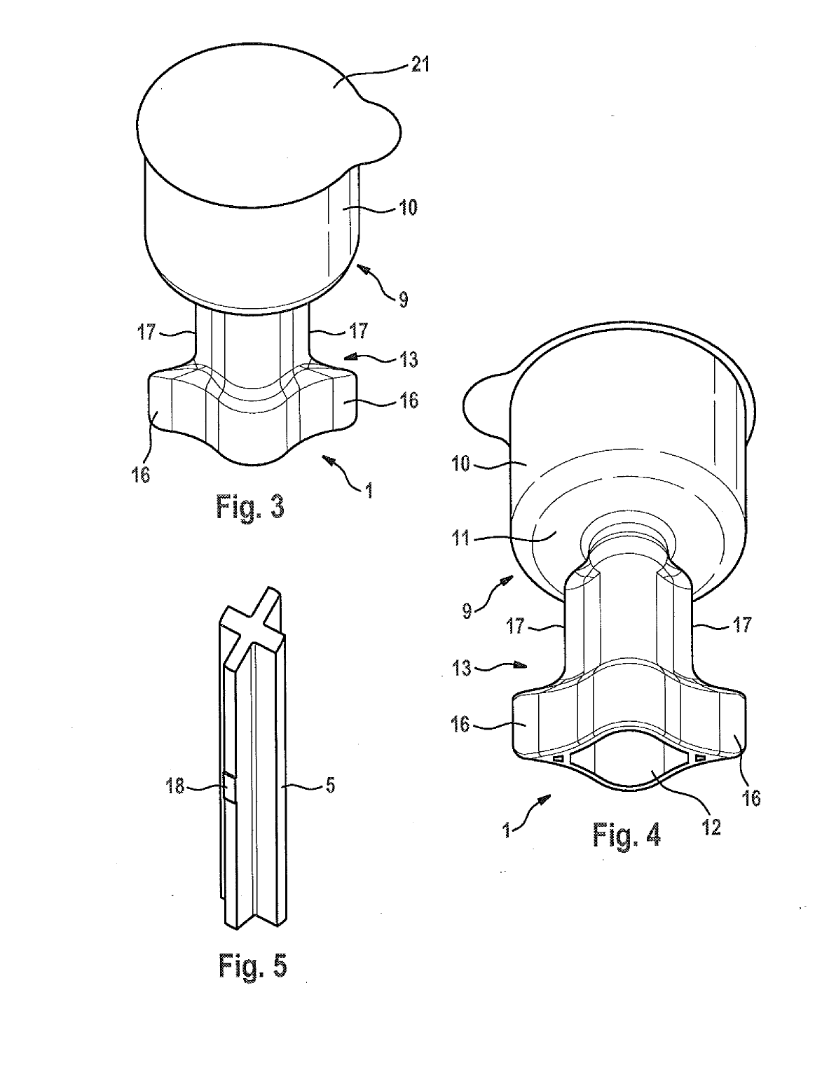

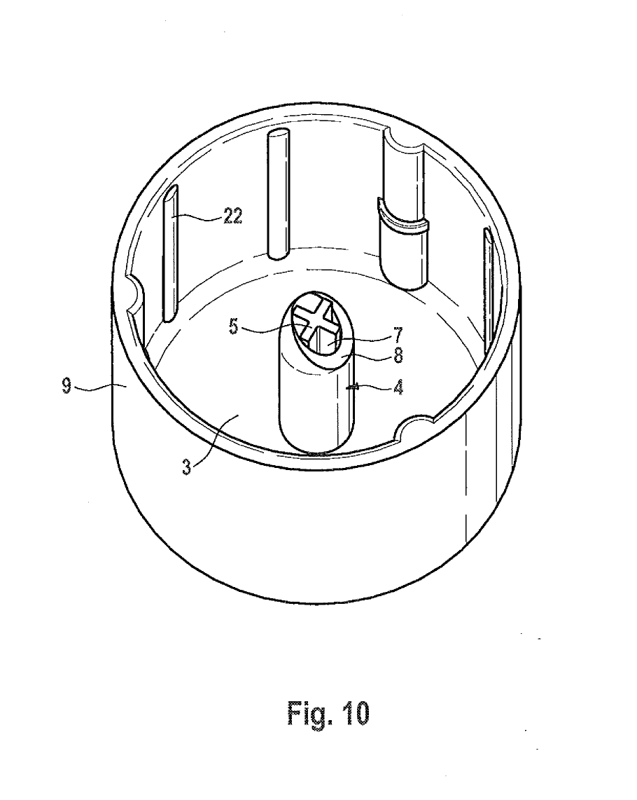

FIGS. 1 to 10 show various views of a first embodiment of a connector 1 according to the invention for containers containing medicinal active ingredient.

The connector 1 comprises a first connection region 3 for the connection of a first container, a guide element 4, a piercing element 5, a partition wall 6 and a second connection region for the connection of a second container. The first connection region 3 is designed here for the connection of a medicinal glass vial. The second connection region 12 is designed here for the connection of a flexible bag. Alternatively, the first connection region 3 and/or the second connection region 12 can of course also be designed for other containers, for example plastic bottles or transfer systems,

The connector 1 comprises a housing 9 with a substantially hollow-cylindrically shaped housing section 10, forming the first connection region 3 and a housing base 11. The guide element 4 is designed as a tubular hollow body with a duct 7 for the transfer of a medicinal active ingredient and is connected to the housing base 11 in the center of the housing base 11. One end of the guide element ends at the housing base 11, while the other end projects into the connection region 3 and thus into the inner region of the housing section 3. The end section 8 of the guide element 4 that projects into the connection region 3 is sharpened or pointed in order to pierce through a membrane 14 of a container 2 to be connected in the connection region 3. The housing section 10 and guide element 4 are arranged axially in a common axis of symmetry 15.

Furthermore, the connector 1 comprises a connecting means 13 which forms the second connection region 12. The connecting means 13 is of tubular design, arranged along the axis of symmetry 15 and is connected by one end to the rear side of the housing base 11 situated opposite to the connection region 3. At the other end, the connecting means 13 comprises two welding lugs 16 formed with mirror-image symmetry for connecting the connecting means to a flexible plastic bag which is not represented in further detail. Furthermore, the connecting means 13 comprises two reinforcing webs 17 extending laterally along the axis of symmetry 15, these webs extending from the base 11 as far as the welding lugs 16 with mirror-image symmetry and having the purpose of reinforcing the connecting means,

The partition wall 6 takes the form of a membrane. It is arranged between the first connection region 3 and the second connection region 12 and, in the closed state, prevents the transport of a medicinal active ingredient between these two regions. Specifically, the partition wall 6 is arranged at the end of the guide element 4 facing away from the first connection region 3 and terminates flush with the rear side of the housing base 11. Alternatively, instead of an arrangement of the partition wall 6 at an end of the duct 7, an arrangement of the partition wall 6 within the duct 7 of the guide element 4 is also possible. Furthermore, the partition wall 6 can be arranged such that it is raised from the housing base 11, for example is arranged in the manner of a cover on the opening of the duct 7. Furthermore, the partition wall 6 can be fastened to the housing base 11 via at least one predetermined breaking point, with the result that the partition wall can be broken open at the at least one predetermined breaking point by the piercing element 5, and the duct 7 is opened in this way. In this case, piercing of the partition wall 6 can be dispensed with.

The partition wall 6 is designed as a wall with a sufficiently thin wall thickness so that it can be pierced and/or broken open by the piercing element 5. In this embodiment, the partition wall is designed in such a way that, after the piercing/breaking-open operation, it remains opened upon a withdrawal of the piercing element 5, which is preferred. It is also possible to design the partition wall 6 in such a way that it can close after a withdrawal of the piercing element 5.

In this exemplary embodiment, the housing 9, guide element 4, partition wall 6 and connecting means 12 are produced in one part and integrally from plastic, here from PP or a blend of PP and SEBS, as an injection molding, with the result that the connector can be produced in a particularly cost-effective manner. Also possible in principle is a multi-part construction and/or a construction composed of a plurality of different materials.

In this embodiment, the piercing element 5 is designed as a solid rod. The piercing element is arranged in the duct 7 and is axially movably guided within the duct 7 by the guide element 4. Between the inner face of the duct 7 and the outer face of the piercing element 5 there is an axially extending clearance which has a sufficient cross section in order to allow medicinal active ingredient to pass through sufficiently quickly between the first connection region 3 and the second connection region 12. Here, the piercing element 5 has a cross-shaped profile which is adapted in its outer dimensions to the inner diameter of the tubular guide element 4, with the result that the piercing element 5 does not tilt. Other profiles can also be used for the piercing element 5, for example profiles with a triangular shape, star shape, square shape and/or cylinder shape. The piercing element 5 can also be designed partially or completely as a hollow body and the transport of the medicinal active ingredient can take place partially or completely through the piercing element 5. Alternatively, the guide element 4 can also be designed with other profiles, for example a square or rectangular profile.

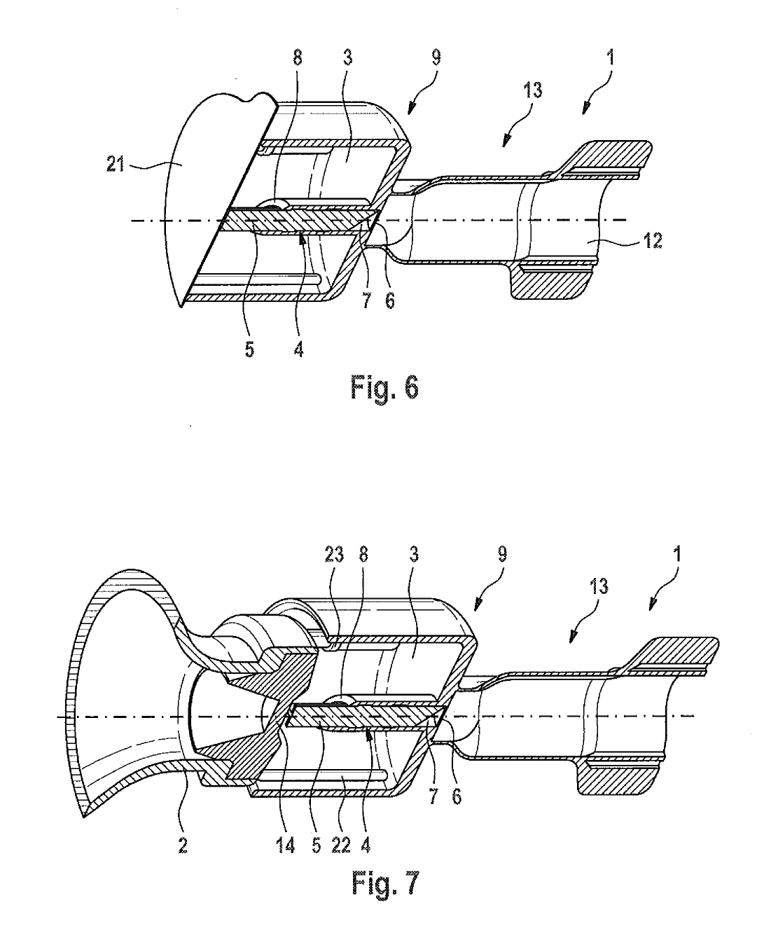

Furthermore, the piercing element 5 is releasably prefixed in a starting position in which the piercing element 5 does not open the partition wall 6. For this purpose, the piercing element 5 comprises two mutually opposite projections 18 which, in the starting position, releasably engage in two corresponding grooves 19 in the guide element 4. By means of sufficient pressure on the piercing element. 5, as can be exerted for example when connecting a glass bottle in the first connection region 3, the piercing element 4 can be released from the starting position and transferred into an end position. When transferring the piercing element 4 from the starting position into the end position, the piercing element 4 opens the partition wall 6; see also FIGS. 6 to 9. The guide element 5 has two further grooves 20 into which the piercing element 4 can latch after opening the partition wall 6 and which prevent the unwanted release of the piercing element 4 from the guide element 4. Alternatively, the piercing element 4 can be provided for example with only one groove or further grooves, and in the case of a plurality of grooves they can be arranged in an offset manner for example. As a further alternative, the piercing element can be prefixed in the guide element by means of an oversize for example.

To make it easier to break open or pierce the partition wall, the piercing element 5 is sharpened or pointed at its end facing the partition wall 6. At its other end facing away from the partition wall 6, the piercing element 5 forms a bearing face in order to take up the pressure over as large an area as possible of a container to be connected and to prevent the piercing element 5 from unwantedly penetrating into the container, for example piercing a rubber stopper closure of the container. For this purpose, the end of the piercing element 5 facing away from the partition wall is designed in this case as a planar face.

In this exemplary embodiment, the piercing element 5 is produced in one part and integrally from a plastic, preferably from PP, PC or PS, as an injection molding

Alternatively, the piercing element 5 can also be multi-part and/or consist of other materials, for example also of a metal, and/or of a plurality of materials.

Furthermore, the connector comprises a removable cover foil 21 which closes the first connection region 3 and protects it from contamination, for example from contamination by means of unintentional contact. The cover foil may, for example, be a manually removable aluminum foil or plastic film.

FIGS. 6 to 9 show the connection of a glass vial 2 to the connector 1.

FIG. 6 shows the connector 1 in the starting state. To connect a glass bottle 2, first of all the cover foil 21 is manually removed. Then, the glass bottle is inserted by its neck into the first connection region 3; see FIG. 7. On inserting the glass bottle 2, the glass bottle 2 exerts an axial pressure on the piercing element 5 on the end of the piercing element 5 projecting beyond the guide element 4 and facing the glass bottle 2, which pressure presses the piercing element 5 by its end facing the partition wall 6 against the partition wall until the piercing element 5 has broken through the partition wall 6 as a result of the pressure. Further insertion of the container 2 causes the piercing element 5 to be axially displaced until the container 2 has reached an upstream container docking position; see FIG. 0. The housing 9 comprises a plurality of axially extending clamping webs 22 which extend on the inner side of the housing section 10 and which connect the glass bottle to the connector 1 by clamping when it is inserted into the connection region. In the container docking position, the membrane 14, here a rubber stopper, arranged in the neck of the container 2 is not yet pierced, such that in this position any transfer of a medicinal active ingredient is still prevented. In particular, the container 2 can be released from the connector 1 without it having already been opened. The latter is particularly advantageous when the container 2 contains expensive active ingredients.

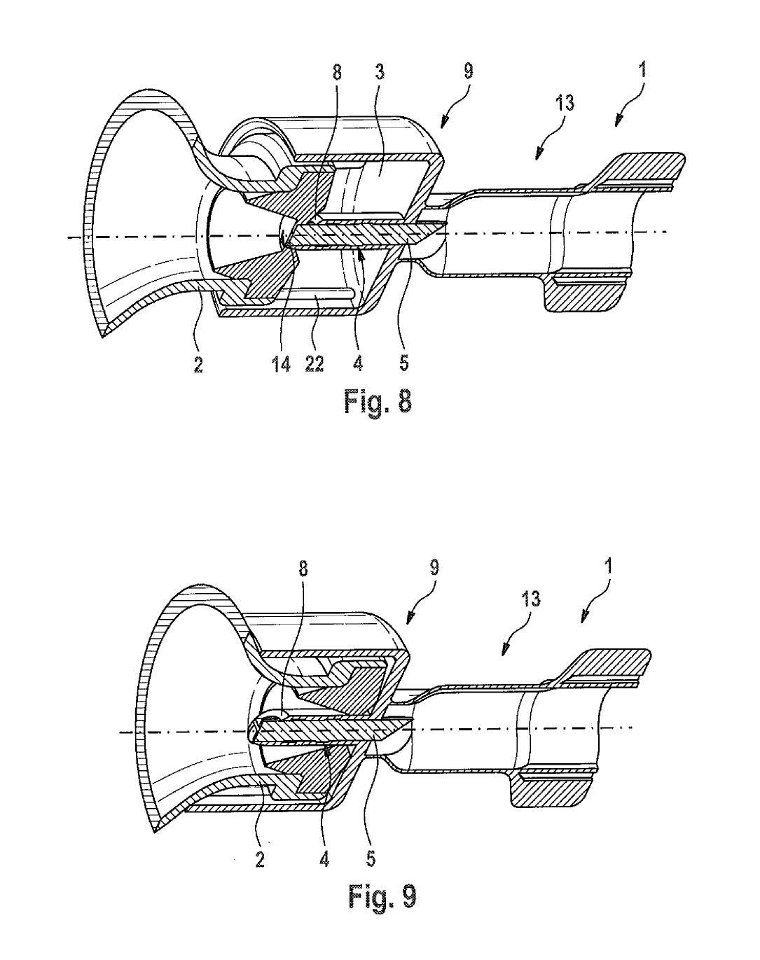

On further inserting the container 2 into the connection region 3, the membrane 14 is pierced by the cannula-like end section 8 of the guide element 4; see FIG. 9. An active ingredient, for example a liquid or a powder, situated in the container 2 can now be transferred by means of the connector 1 and for example diluted and/or dissolved, for example in a flexible bag connected to the connecting means 13, or, conversely, an active ingredient can be passed into the container 2. On further inserting the container 2, the container 2 reaches an end position in which one or more projections 23 on the inner side of the housing section 10, see for example FIG. 8, positively engage behind the container 2 at its neck and prevent the container 2 from slipping out of the first connection region 3 of the connector 1.

The connection region 3 can be provided with a further undercut, for example by means of additional projections, which already fixes the container 2 in the docking position with the formation of a positive fit. The clamping webs 22 can also be present in this variant; alternatively, however, they can also be dispensed with. Such a second undercut facilitates the connection of containers having different dimensions to the connector 1.

The container 2 can be premounted on the connector in the docking position. The premounted container 2, the connector 1 and a container, in particular a bag, connected to the connector 1 can be enclosed as a set in an outer bag. This makes it possible to reliably mix the components outside the laminar flow region without the risk of contamination.

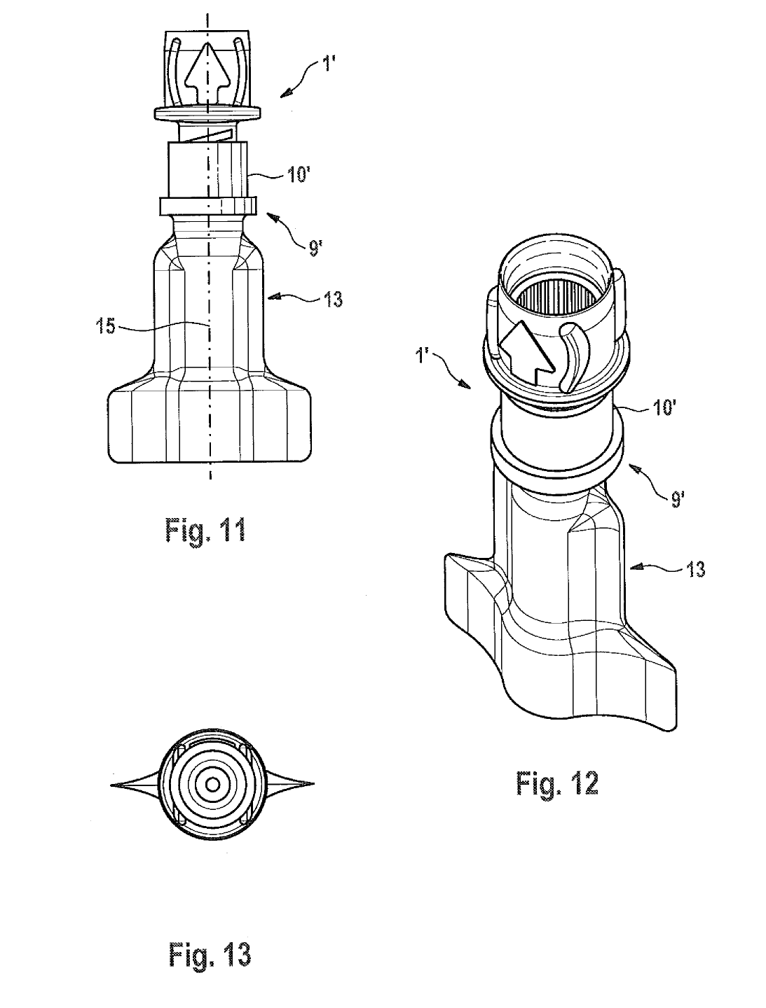

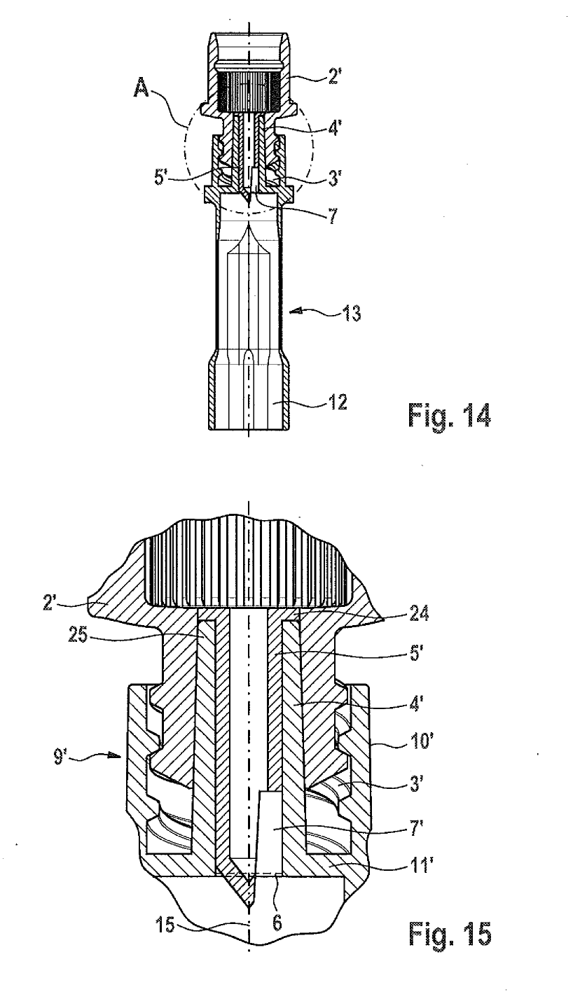

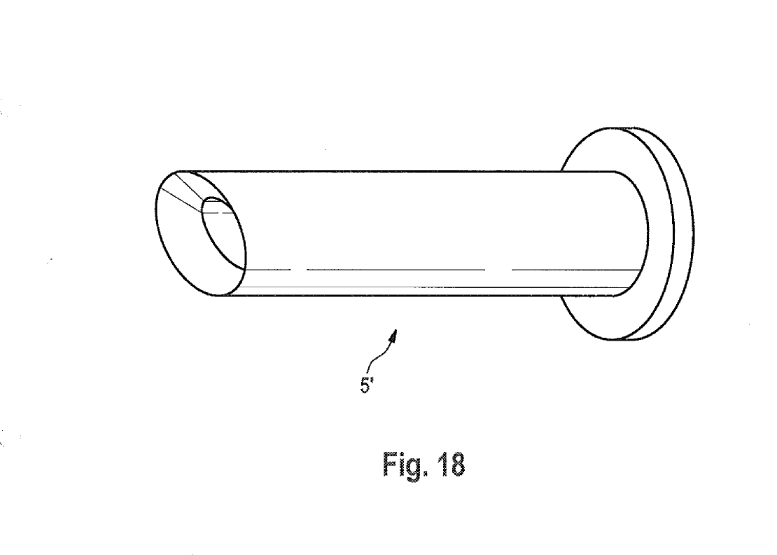

FIGS. 11 to 18 show a second embodiment of a connector 1′ according to the invention for containers containing medicinal active ingredient, in various views. Elements or components which correspond to those of the first embodiment have been designated with the same reference signs which were used to designate the components of the first embodiment,

The connector 1′ according to the second embodiment comprises a first connection region 3′, a guide element 4′, a piercing element 5′, a partition wall 6 and a second connection region 12. The second connection region 12 is formed by a connecting means 13 which corresponds to that of the first embodiment. As a departure from the first embodiment, the first connection region 3′ is designed as a Luer connection of a Luer lock (ISO 594/1), here as a Luer male part. Connection region 3′, guide element 4′, piercing element 5′ and connecting means 13 are arranged along the common axis of symmetry 15.

The connector 1′ comprises a housing 9′ having a substantially cylindrical housing section 10′ with an inner thread and a housing base 11′ which form the connection region 3′. The guide element 4′ is designed as a hollow outer cone and projects into the connection region T. By being designed as a hollow body, the guide element 4′ comprises an internal duct 7 which is provided for the transfer of a medicinal active ingredient. The guide element 4′ is connected to the housing base 11′ in the center of the housing base 11′.

The partition wall 6 is arranged in the plane of the housing base 11′ in a corresponding manner to the partition wall 6 according to the first embodiment and correspondingly designed. In the closed state, the partition wall 6 prevents any transport of a medicinal active ingredient between the first connection region 3′ and the second connection region 12.

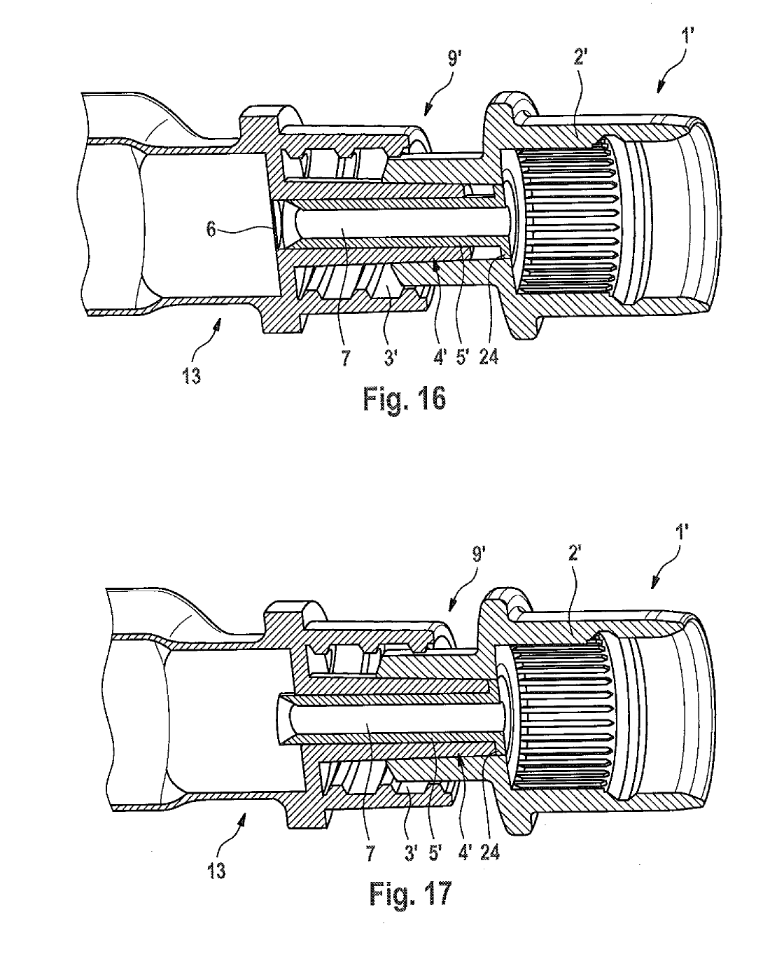

The processing element 5′ is designed to be tubular as a hollow body. The piercing element 5′ is arranged in certain sections in the duct 7 and is axially movably guided within the duct 7 by the guide element 4′. In the guided section, the outer diameter of the piercing element 5′ is tailored to the inner diameter of the guide element 4′ to obtain optimally play-free mounting. At its end facing the first connection region 3′, the piercing element 5′ has a collar 24, see FIG. 5, while at its end facing away from the first connection region 3′, the piercing element 5′ is sharpened or pointed in order to cut more easily through the partition wall 6. In a starting position, in which the piercing element 5′ does not open the partition wall 6, see FIG. 16, the collar 24 is spaced from the guide element 4′, while in an end position, in which the piercing element 5′ opens the partition wall 6 in order to transfer a medicinal active ingredient, the collar 24 rests on the upper edge 25 of the guide element 4′, see FIGS. 15 and 17, with the result that a further insertion of the piercing element 5′ into the guide element 4′ is blocked. Alternatively, the piercing element 5′ can also be designed as a solid body, for example with a cross-shaped, star-shaped, triangular or similar profile. In this case, a clearance remaining between the inner face of the guide element 4′ and the outer face of the piercing element 5′ can be used for the transport of the medicinal active ingredient.

In this exemplary embodiment, housing 9′, guide element 4′, partition wall 6 and connecting means 6 are produced as a one-part and integral injection molding from a plastic, preferably PP or a PP-SEBS blend. It would also be possible in principle to have a multi-part construction and to use other and/or different materials. The piercing element 5′ is likewise a one-part and integral injection molding made of plastic, preferably PP, PC or PS. As an alternative, it would also be possible here in principle to have a multi-part construction and to use other and/or different materials.

In addition to the connector the depicted figures show an element of a container, here a Luer female part 2′, which is connected or has been connected to the connector 1′. The Luer female part 2′ can be a constituent part of a syringe or a transfer system, for example.

FIG. 16 shows the state after attaching the Luer female part 2′ and before locking the Luer female part 2′ with the Luer male part of the connector 1′. In this position, the guide element 4′ together with the projecting piercing element 5′ is already partially inserted into the inner cone of the Luer female part 2′. The outer diameter of the collar 24 of the piercing element 5′ is tailored to the diameter of the inner cone of the Luer female part 2′ such that the collar 24 cannot be plugged completely through the inner cone, but is clamped in at its outer edge beforehand by the tapering inner cone, here virtually at the end of the inner cone. When locking the Luer female part 2′ with the male part, which is formed by the housing 9′, the Luer female part 2′ is displaced axially in the direction of the connector 1′. During this movement, the piercing element 5′ clamped at the collar 24 is taken along by the female part 2′, and the partition wall 6 is broken open or pierced and opened by the sharpened or pointed end of the piercing element 5′, see FIG. 17. The connection between the first connection region 3′ and the second connection region 12 is now opened, thereby allowing the transport of a medicinal active ingredient between a flexible bag connected to the connector 1′ in the connection region 12 or another container with a container connected by the Luer female part 2′.

To protect against contamination and/or to protect against unwanted opening of the partition wall 6 by a movement of the piercing element 5′, the connection region 3′ of the connector 1′ with piercing element 5′ can be covered by a manually removable cap (not shown). Furthermore, the piercing element 5′ is preferably prefixed in its extended starting position, for example by means of a tongue-and-groove connection in a similar manner to that of the first embodiment. It is also possible to fix the piercing element 5′ in the end position, for example in order to prevent the piercing element 5′ from falling out when releasing the female part 2′.

The connectors 1, according to the invention have the advantage that they can be produced cost-effectively. Furthermore, a sterile connection between two containers can be produced manually in a simple manner both reliably and quickly. In order to quickly transfer a medicinal active ingredient, guide element 4, 4′ and piercing element 5, 5′ can be designed with suitable profiles.

What is claimed:

1. A connector for containers containing medicinal active ingredient, comprising:
a first connection region for the connection of a first container, the first connection region including:
a guide element having a duct designed for transferring a medicinal active ingredient and having first and second ends;
a piercing element arranged, at least in certain sections, in the duct and axially moveably guided within the duct by the guide element; the piercing element having first and second ends, the second end including a stop, and
a partition wall located proximate the first end of the guide element;
wherein, by connecting the first container in the first connection region, the piercing element can be moved from a starting position, in which the piercing element does not open the partition wall, into an end position, in which the first end of the piercing element opens the partition wall in order to transfer a medicinal active ingredient, and wherein the stop on the second end of the piercing element engages with the second end of the guide element to limit further movement of the piercing element.

2. The connector as claimed in claim 1, wherein the first end of the piercing element is sharpened or pointed and wherein the stop on the second end of the piercing element is in the form of a collar.

3. The connector as claimed in claim 1, wherein the piercing element projects by its first end facing the first connection region beyond the guide element.

4. The connector as claims in claim 1, wherein the piercing element and the guide element are designed in such a way that a clearance for transferring a medicinal active ingredient remains between an outer face of the piercing element and an inner face, forming the duct, or the guide element.

5. The connector as claimed in claim 1, wherein the piercing element is designed as a solid body and/or as a hollow body.

6. The connector as claimed in claim 1, wherein the piercing element consists at least partially of a plastic.

7. The connector as claimed in claim 1, wherein the partition wall is arranged within the duct or at an end of the duct.

8. The connector as claimed in claim 1, wherein the partition wall is arranged at the end of the piercing element facing away from the first connection region.

9. The connector as claimed in claim 1, wherein the piercing element is releasably prefixed in the starting position.

10. The connector as claimed in claim 1, wherein the connector is designed such that, when connecting to the first connection region and transferring the container into an upstream container docking position, at first the partition wall is opened by the piercing element.

11. The connector as claimed in claim 1, additionally comprising a housing with a housing section, forming the first connection region, and a housing base, wherein the guide element is connected to the housing at the housing base.

12. The connector as claimed in claim 1, additionally comprising a second connection region for the connection of a second container, wherein the partition wall is arranged such that in the closed state it prevents the transport of a medicinal active ingredient between the first connection region and the second connection region.

13. The connector as claimed in claim 12, wherein the second connection region is formed by a connector connected to the housing base.

14. The connector as claimed in claim 13, wherein the housing and the connector are one-part.

15. The connector as claimed in claim 1, wherein the first connection region is designed for the connection of a glass bottle or as a Luer connection.

16. The connector as claimed in claim 1, wherein the piercing element consists completely, of a plastic.

17. The connector as claimed in claim 1, wherein the piercing element consists at least partially of polypropylene, polycarbonate or polystyrene.

18. The connector as claimed in claim 14, wherein the housing and the connector are integral.

19. A connector for containers containing medicinal active ingredient, comprising:
a first connection region for the connection of a first container, the first connection region including:
a housing having a housing base;
a guide element connected to the housing base and having a duct designed for transferring a medicinal active ingredient;
a piercing element arranged, at least in certain sections, in the duct and axially moveably guided within the duct by the guide element, and
a partition wall located in the housing base and proximate a first end of the guide element;
a second connection region for the connection of a second container, the second connection region including a connector connected to the housing base;
wherein, by connecting the first container in the first connection region, the piercing element can be moved from a starting position, in which the piercing element does not open the partition wall thereby preventing transfer of the medicinal active ingredient between the first container and the second container, into an end position, in which the first end of the piercing element opens the partition wall in order to allow transfer the medicinal active ingredient between the first container and the second container.
